(19) United States
(12) Patent Application Publication
(10) Pub. No.: US 2012/0318282 A1
(43) Pub. Date: Dec. 20, 2012

(54) General theory of equilibrium

(75) Inventor: Anthony Austen White, Spring Hill Brisbane (AU)

(73) Assignee: ANTHONY AUSTEN WHITE

(21) Appl. No.: 13/199,389

(22) Filed: Aug. 29, 2011

(30) Foreign Application Priority Data
Jun. 17, 2011 (AU) 2011202910

(51) Int. Cl. A61B 19/00

(52) U.S. Cl. 128/897

(57) ABSTRACT

N.B. “Theory” refers to the General Theory of Equilibrium

Number (e.g. 001, 002) refers to paragraphs in the description of the theory.

The theory is an original physics theory dealing with vibrational movement, of matter molecules, and energy (radiation). The theory discloses that (a) at universal creation matter molecules created energy with an equal frequency to its molecular frequency. Molecular frequency is referred to as mechanical force (M.F.). Should one prefer, one could refer to it as gravitational force (G.F.). Energy is referred to as Hertz Frequency Force (H.F.) or electromagnetic (E.H.F.) (001, 002, 003, 004, 005, 006). Today and forever more matter per medium of the energy it omits seeks equilibrium with other matter (007) (b) it has pursuant maths (008) (c) the (M.F. or G.F.) of matter, can be varied thereby enabling the curing of disease, thus providing humanity with a new basis for medicine. The procedure to so vary is described (009, 010, 011, 011(a), 011(b), 011(c), 011(d), 011(e), 011(f), 012, 013, 015). The Theory unifies or equates mechanical or gravitational force (M.F. or G.F.) with electromagnetic force (E.H.F.) (014).

From Brownian movement days science has known that molecules of matter vibrate. What science does not know is that molecular vibration leads to radiation vibration and that radiation vibration leads to the molecular vibration.

For a single piece of matter left in isolation molecular vibration=radiation vibration i.e. Rate of molecular vibration=rate of radiation vibration.

Calling rate of molecular vibration the mechanical force of matter and the rate of radiation vibration the Hertz frequency force of radiation M.F.=H.F. whereas M.F. stands for Mechanical Force and H.F. stands for Radiation Force.

Hertz is the name given by science for radiation frequency. There is no name given to mechanical force. When there are 2 or more pieces of matter the M.F. and the H.F. interact. The M.F. leads H.F. and the H.F. leads to the M.F. One determines the other. It is ongoing for the life of the universe which I believe will never end.

Just with Human Beings. Human Beings are Experienced and Consciousness Beings. Consciousness leads to Experience and Experience leads to Consciousness. It is ongoing for the life of a Human. Consciousness is Electromagnetic and Experience is Mechanical. H.F. to M.F. or M.F. to H.F. to M.F. Those functions of vibrations go on for ever and ever.

H.F. and M.F. are two sides of one coin. The coin being the movement of matter. When the radiation is electromagnetic the formula becomes M.F.=−E.H.F. The E standing for Electromagnetic. There are 92 elements in the Universe. The rule is the higher their respective density, the higher their M.F. It is a question of relativity.

Matter via E.H.F. seeks equilibrium. The fast leads to the fast. The slow leads to the slow. From interaction we can see that if an E.H.F. is relatively faster than the M.F. if a piece if matter it plays on, the M.F. of the piece of matter will go faster. Conversely, of E.H.F. relatively slower the M.F. will go slower.

E.H.F.=M.F.; M.F.=E.H.F. Pursuantly to make the maths easier you can use one for the other. The % increase becomes incoming E.H.F.—original E.H.F. for any point of time. The % decrease for any point of time becomes original E.H.F.—incoming E.H.F. “Then divide the respective increase or decrease answer by its respective original E.H.F. and multiply by 100”.

Bacteria, viruses and germs create themselves, Electromagnetism enables them, to create themselves. Bacteria, viruses and germs are the cause of most disease, illness and ailment. Fluctuations in E.H.F. are the cause of most disease, illness and ailments. Such as fluctuations in E.H.F. can also cure most disease, illness and ailments.

Soft matter has a lower density M.F. than hard matter. Fast is hard, slow is soft. Pursuantly, if you play fast E.H.F. onto a relatively slower M.F. (soft tissue) the soft tissue will go harder (its M.F. will increase). Conversely, playing a slow E.H.F. onto a relatively hard tissue will make it go softer. Therein lies the curing of most disease, illness of ailments by direct application of E.H.F., it's as simple as that.

The aim has been to straight out kill the viruses, bacteria or germs or destroy them by destroying their environment with the direct application of E.H.F.

From the above it can be seen that as soft diseased tissue (low M.F. rate) is relatively softer than a previous soft diseased tissue (low M.F. rate) than a higher frequency (E.H.F.) has to be chosen to get the diseased tissue to return to the desired state ie. vibrating (M.F.) or the same rate as before diseased. One may, or may not have to go up the frequency ladder in steps to avoid damage to other body parts and tissue.

It is not a question of how much of a given frequency, but rather of how high the frequency (E.H.F) has to be to destroy the germs, bacteria or disease.

It should be the noted that very high electromagnetic frequency range waves will destroy most, if not all, germs, bacteria or viruses as they will not be able to escape the vibration force of the said electromagnetic frequencies. Whereas, the distance between the trough and crest of lesser frequency waves may enable the said bugs to escape. The inverse of that said distance determines the vibration force of a given frequency. The law is the greater the said distance, the lesser the said force; the lesser the said distance, the greater the said force.

A very short spurt of frequency (E.H.F.) in the very high electromagnetic frequencies rage should destroy most, of not all, of the germs, bacteria or viruses.

A low range frequency (E.H.F. has to be used to reduce swelling or inflammation (high M.F., Mechanical Force, material vibration force) thereby taking the pressure off of nerves and the relieving of pain. The objective is to return body parts or tissue to their normal rate of M.F.

The danger with both high and low is that it will make surrounding body parts or tissues, or for internal disease or pain, body parts or tissue in the path of the radiation transmission go permanently harder or softer. The authors thinking is that it wont, that the said body parts or tissue will retain or recover their original normal) M.F. in a brief period of time after a short burst of energy (radiation E.H.F.), that for internal injuries the transmitted radiation will seek out the disease or pain as if there is a God.

Now to pharmaceuticals, the curing of most diseases, illness or ailments in the

Universe by M.F. For a disease, illness or ailment which is relatively soft to its surrounds, low M.F. (low density, low vibration rate) formulate a compound which is higher M.F. (higher density, higher vibration rate). The illness, disease or ailment thereby becomes the ham in the sandwich, between the healthy body tissues and the medication (compound). It gets a double dose. By one side, healthy tissue (which is harder, higher density and therefore emitting a higher E.H.F.) and the other side, the compound, which is also harder and therefore also emits higher E.H.F.

Matter seeks equilibrium. Therefore both sides “attack” the “ham” and destroy it. The reverse is true for a problem which is relatively hard to its surroundings. Then a soft low density compound is required. The “ham in the sandwich” is repeated with the same result. Once again the aim has been directly kill, or indirectly, by destroying the environment of bacteria, or viruses or germs.

The General Theory of Equilibrium unifies molecular vibrations with radiation vibrations thereby giving Humanity the basis for the curing of most diseases, illnesses or ailments from here to eternity.

In this specification the author has gone into the why's and how's. The explicit, precise, how's; what precise level of E.H.F is to be “directed”, as aforesaid, or the precise compounds is left to scientists and chemicals internationally. I have no doubt to their brilliance. No doubt that they will achieve on the basis of the procedure of the General Theory of Equilibrium. The General Theory of Equilibrium is also suited to Environmental questions. It is also suited to General questions involving “hot” or “cold” or “hard” or “soft”, or “fast” or “slow”.

1) The General Theory of Equilibrium as described is not known to Mankind and is not obvious to them. It is my original Individual, Intellectual, Property Right. A contribution of mine to Humanity.

2) The said theory is a General Theory. I am making no claims as to specifics. I am not trying to Patent specifics e.g. That electromagnetic frequency (a) will cure or relieve diseases, or illness, or ailments (b) Nor am I trying to Patent that pharmaceutical (a) will cure or relieve disease or ailment (b) Nor am I trying to patent specifics in other fields which the description of the said theory applies to. The said Theory is particularly appropriate for medicine and the environment. The General Theory of Equilibrium will inspire and enable others to achieve and make contributions to Humanity re specifics, their basics of said being the description of the said Theory. Those others may be self employed or a Company which organises labour. These others will be able to make monetary profit. Their profit is partly due to their skills and brilliance and partly due to my Individual, Intellectual, Property Right. An individual is His or Her Individual, Intellectual, Property Right. It is His or Her life. Without it He or She exists as a slave with all of its consequences. Pursuantly, I feel that I am entitled to Royalties when my Individual, Intellectual, Property Right is utilised by others to make monetary profit e.g. $2.00 per prescription, or $2.00 for directly using electromagnetics to relieve or cure disease, illness or ailment. The environment is a different matter. Should Humanity be able to utilise me via the General Theory of Equilibrium to achieve or partly achieve an impossible dream; namely to moderate the weather, and more equally distribute the Worlds rainfall it will be worth billions and trillions to Humanity and the Royalties asking price will be very substantial. What I wish to Patent is an original, Individual Intellectual, Property Right of mine; namely the General Theory of Equilibrium with its methodical description of the basic whys and hows of Equilibrium.

3) The sizes of bacteria and viruses are not known to man. It is known that they are very, very small. It is claimed that electromagnetic frequencies in the very high range of electromagnetic frequencies e.g. radio waves of very, very high frequency, will destroy most, if not all, bacteria and viruses as they will not be able to escape the vibration force of such said frequencies.

4) It is claimed that low level E.H.F. (frequencies) will cause the M.F. (Mechanical Force, tissue or body part vibration rate) to go slower, thereby reducing swelling or inflammation and thus taking the pressure off of nerves and consequently relieving pain.
